(19) United States
(12) Patent Application Publication
(10) Pub. No.: US 2016/0084769 A1
(43) Pub. Date: Mar. 24, 2016

(54) Fluorescent Caffeine Sensor And Portable Kit And Microfluidics Device For Caffeine Detection

(71) Applicants: NATIONAL UNIVERSITY OF SINGAPORE, Singapore (SG); ULSAN NATIONAL INSTITUTE OF SCIENCE AND TECHNOLOGY, Uljugun, Ulsan (KR)

(72) Inventors: Young-Tae Chang, Singapore (SG); Wang Xu, Singapore (SG); Duanting Zhai, Singapore (SG); Yoon-Kyoung Cho, Uljugun, Ulsan (KR); Tae-Hyeong Kim, Uljugun, Ulsan (KR)

(21) Appl. No.: 14/785,468

(22) PCT Filed: Apr. 17, 2014
(86) PCT No.: PCT/SG2014/000167

§ 371 Date: Oct. 19, 2015

Related U.S. Application Data
(60) Provisional application No. 61/813,684, filed on Apr. 19, 2013.
(60) Provisional application No. 61/845,560, filed on Jul. 12, 2013.

(51) Int. Cl. G01N 21/76; G01N 33/02; G01N 1/40; G01N 21/78

(52) U.S. Cl. CPC G01N 21/76; G01N 21/78; G01N 33/02; G01N 1/405

(57) ABSTRACT

The present invention relates to Caffeine Orange, a novel aqueous-phase fluorescence turn-on sensor for caffeine that is structurally based on a BODIPY-scaffold. The present invention further provides for methods of detecting and measuring caffeine in aqueous media. A change in the intensity or visible color of the fluorescence is detectable by either a fluorimeter or by the naked eye. The methods disclosed herein provide for the utilization of a reverse-phase SPE column, optionally as a component in a syringe or a microfluidics-based automation detection system. The invention further provides for the solid phase extraction of an analyte such as caffeine from a liquid medium, the extraction occurring on a microfluidic disc.

### RELATED APPLICATIONS

This application claims the benefit of U.S. Provisional Application No. 61/813,684, filed on Apr. 19, 2013 and U.S. Provisional Application No. 61/845,560, filed on Jul. 12, 2013. The entire teachings of the above applications are incorporated herein by reference.

### BACKGROUND OF THE INVENTION

Caffeine, a type of alkylated oxopurine, is one of the most frequently consumed alkaloids. In natural sources it mainly exists in commonly consumed food or drinks such as coffee, black tea and cocoa beans. It is found in a broad spectrum of consumer products that include soft drinks and analgesics, and functions as an important central nervous system stimulant. In spite of its effect in nerve stimulation, it exhibits significant adverse impact on children and pregnant women. Therefore a convenient and reliable system for the determination of caffeine in a consumable good is desirable and marketable.

Chromatographic techniques such as gas chromatograph (GC), high pressure liquid chromatography (HPLC) and capillary electrophoresis (CE) are among the standard methodologies for the quantitative measurement of caffeine, in various formulations. Although the measurement time has been shortened to within a few minutes, its intrinsic analysis mode leaves no room for on-line detection.¹ Chemosensors, on the other hand, represents a newly-emerging and fast-developing field and presents a solution for this issue. Since the first artificial receptor for caffeine developed by Waldvogel et al. appeared in 2000, several synthetic caffeine receptors were also reported. However, many of these synthetic caffeine receptors lack easily-detectable or practically-applicable responses towards the binding event.²

Due to its potential for high sensitivity and simple handling, fluorescence has been a widely utilized technique in many fields, such as biological analyses, chemical detection and environmental monitoring, etc. Small molecule fluorescence chemosensors, which are selective towards a target substance or biological phenomenon have also evolved for several decades and are now used in various detection processes.³

One recent aqueous phase fluorescent caffeine detection method was reported by using a commercially available dye. Through fluorescence turn-off mode, the dye was used to estimate the caffeine amount in several drinks and medicines quantitatively, along with a fluorimeter. Although it proved its feasibility in quantitative caffeine measurement, its usage is limited by the fluorescence turn-off property, which renders it practically inapplicable in real-life detection. Hence, there is a need to develop a caffeine sensor having practical applicability through, for example, aqueous phase fluorescence turn-on.² Furthermore, methods are needed that enable such a caffeine sensor to be used with portability, reliability, and minimal operation time.

### SUMMARY OF THE INVENTION

The invention is based on the discovery of a novel fluorescence turn-on probe for the detection of analytes such as caffeine.

In one aspect, the invention relates to kits for the detection of caffeine in a sample, comprising a reverse phase solid phase extraction column, a compound having the structure of Formula (I):

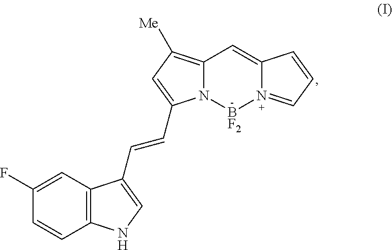

or a salt thereof; and instructions indicating the use of the kit for the detection of caffeine in a sample. In certain embodiments, the kits further comprise a light source having a wavelength of about 532 nm. In further embodiments, the reverse phase solid phase extraction column is enclosed in a syringe.

In another aspect, the invention is a compound having the structure of Formula (I), or a salt thereof.

In another aspect, the invention relates to methods for the fluorescence-based selective detection of caffeine in a liquid medium, comprising the steps of (a) loading a solid phase extraction column with a sample of a liquid medium thought to contain caffeine, such that caffeine, if present, is retained on the column and one or more impurities, if present, pass through the column; (b) contacting the solid phase extraction column loaded with the sample with one or more solutions sufficient to elute a solution thought to contain caffeine off of the column; (c) contacting the solution thought to contain caffeine of step (b) with a compound of Formula (I) or a salt thereof to form an incubation media; (d) incubating the media of step (c) for a period of time sufficient to enable detection of caffeine by fluorescence if present in the solution; and (e) detecting fluorescence in the incubated media. The presence of caffeine in the liquid medium is indicated by a change in fluorescence signal as compared to a fluorescence signal of the compound of Formula (I) not in the presence of the solution thought to contain caffeine.

In certain embodiments, detecting fluorescence in the incubated media comprises qualitative visual analysis or analysis by fluorescence reader, fluorescence meter or fluorescence spectroscopy.

In other embodiments, the change in fluorescence comprises a change in the color of the fluorescence. In certain embodiments, the change in the color of the fluorescence is detectable under visible light or a wavelength portion thereof or ultraviolet light.

In another embodiment, the presence of caffeine in the liquid medium is indicated by an orange-colored fluorescence when under irradiation with a light source having a wavelength of about 532 nm.

In other embodiments, the change in fluorescence comprises a change in fluorescence intensity. In particular embodiments, the change in fluorescence intensity comprises an increase in fluorescence intensity.

In certain embodiments, the solid phase extraction column is enclosed in a syringe, and in alternate embodiments, it is a component of a microfluidics device.

In another aspect, the invention relates to methods for solid phase extraction of an analyte from a liquid medium on a microfluidic disc, comprising the steps of (a) providing a rotatable microfluidic disc, the disc comprising: a sample inlet, an extraction chamber comprising a solid phase extraction column, wherein an upstream end of the solid phase extraction column is in fluid communication with the sample inlet, and a sample outlet, wherein a downstream end of the solid phase extraction column is in fluid communication with the sample outlet, and further wherein the sample outlet is disposed at a greater distance from the spinning axis of the rotatable disc than the sample inlet; (b) loading a liquid medium thought to contain an analyte into the sample inlet; and (c) rotating the disc such that centrifugal force causes the liquid medium to travel from the sample inlet through the solid phase extraction column into the sample outlet, such that the analyte, if present, is retained on the column, wherein liquid flow through the solid phase extraction column occurs in a direction perpendicular to the direction of radial force.

In certain embodiments, the disc further comprises an upper disc plate, a lower disc plate, wherein the solid phase extraction column is oriented between the upper and lower disc plates such that a liquid passing therethrough travels in a direction perpendicular to the plane of the upper and lower disc plates, optionally a serpentine microfluidic channel, wherein a downstream end of the solid phase extraction column is in fluid communication with the serpentine channel, and further wherein a downstream end of the optional serpentine microfluidic channel is in fluid communication with the sample outlet.

In further embodiments, the disc comprises one or more reagent chambers containing a reagent liquid, each independently selected from a pre-washing buffer, a salt buffer, a washing buffer, an elution buffer, a blocking buffer or a detection solution.

In further embodiments, the methods comprise the step of eluting the analyte from the solid phase extraction column by contacting the column with an elution buffer, wherein the step of eluting is performed after step (c).

In further embodiments, the methods comprise controlling flow resistance by directing liquid flow through the serpentine channel, thereby altering the elution time of the analyte into the sample outlet.

In certain embodiments, the solid phase extraction column comprises reverse-phase hydrocarbon-functionalized silanes, glass membranes, silica beads or polymer beads.

In some embodiments, the analyte is caffeine.

In another aspect, the invention relates to methods for fluorescence-based selective detection of an analyte in a liquid medium on a microfluidic disc, the method comprising the steps of (a) providing a rotatable microfluidic disc, the disc comprising an upper disc plate; a lower disc plate; a sample inlet; one or more reagent chambers, each independently containing a reagent liquid, an extraction chamber comprising a solid phase extraction column; wherein an upstream end of the solid phase extraction column is in fluid communication with the sample inlet and the one or more reagent chambers, and further wherein the solid phase extraction column is oriented between the upper and lower disc plates such that a liquid passing therethrough travels in a direction perpendicular to the plane of the upper and lower disc plates; one or more serpentine microfluidic channels, wherein a downstream end of the solid phase extraction column is in fluid communication with the one or more serpentine channels; a waste chamber, wherein the waste chamber is disposed at a greater distance from the spinning axis of the rotatable disc than the sample inlet, and wherein the waste chamber is in fluid communication with the downstream end of a serpentine microfluidic channel; and a detection chamber, wherein the detection chamber is disposed at a greater distance from the spinning axis of the rotatable disc than the sample inlet, and wherein the detection chamber is in fluid communication with the downstream end of a serpentine microfluidic channel. The detection chamber contains a fluorophore of the structure of Formula

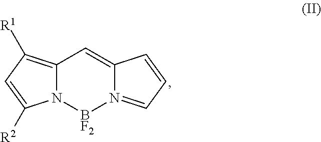

or a salt thereof;
wherein R¹ is C₁-C₁₂ alkyl; and
R² is C₁-C₆ alkyl or C₂-C₆ alkenyl, optionally substituted with C₆-C₁₄ aryl or C₃-C₁₃ heteroaryl.

The method further comprises (b) loading a liquid medium thought to contain the analyte into the sample inlet; (c) rotating the disc such that centrifugal force causes the liquid medium to travel from the sample inlet through the solid phase extraction column into the sample outlet, such that the analyte, if present, is retained on the column, and one or more impurities, if present, pass through the column and into the waste chamber, wherein liquid flow through the solid phase extraction column occurs in a direction perpendicular to the direction of radial force; (d) contacting the solid phase extraction column with one or more reagent liquids from one or more reagent chambers, wherein at least one of the one or more reagent liquids is sufficient to elute a solution thought to contain the analyte off of the column; (e) contacting the solution thought to contain the analyte of step (d) with the fluorophore of Formula (II) in the detection chamber to form an incubation media; (f) incubating the media of step (e) for a period of time sufficient to enable detection of the analyte by fluorescence if present in the solution; and (g) detecting fluorescence in the incubated media, wherein a change in fluorescence signal as compared to fluorescence of the fluorophore of Formula (II) not in the presence of the solution thought to contain the analyte is indicative of the presence of the analyte in the liquid medium.

In certain embodiments, detecting fluorescence in the incubated media comprises qualitative visual analysis or analysis by fluorescence reader, fluorescence meter or fluorescence spectroscopy.

In some embodiments, change in fluorescence signal is a change in the color of the fluorescence. In certain embodiments, the change in the color of the fluorescence is detectable under visible light or a wavelength portion thereof or ultraviolet light.

Alternately, the change in fluorescence signal is a change in fluorescence intensity. In some embodiments, the change in fluorescence intensity is an increase in fluorescence intensity.

In certain embodiments, the one or more reagent chambers each contain a reagent liquid, each independently selected from a pre-washing buffer, a salt buffer, a washing buffer, an elution buffer, a blocking buffer or a detection solution.

In some embodiments, the methods further comprise controlling the flow resistance by directing liquid flow through the serpentine channel, thereby altering the elution time of the caffeine into the sample outlet.

In some embodiments, the solid phase extraction column comprises reverse-phase hydrocarbon-functionalized silanes, glass membranes, silica beads or polymer beads.

In further embodiments, a path through which the liquid medium flows is manipulated by an actuation of at least one valving unit.

In further embodiments, the flow of the reagent liquid is manipulated by an actuation of at least one valving unit. In certain embodiments, the valving unit comprises a phase transition valve that is actuated by laser irradiation or heat. In particular embodiments, the phase transition valve comprises ferrowax, hydrogel, sol-gel, ice or a polymer film.

In certain embodiments, the analyte is caffeine.

In certain embodiments, wherein the fluorophore of Formula (II) is a compound having the structure of Formula (I) or a salt thereof.

In particular embodiments, under irradiation with a light source having a wavelength of about 532 nm, an orange-colored fluorescence is indicative of the presence of caffeine in the liquid medium.

In another aspect, the invention is a centrifugal microfluidic device, comprising an upper disc plate; a lower disc plate; a sample inlet; one or more reagent chambers, each independently containing a reagent liquid; an extraction chamber comprising a solid phase extraction column, wherein an upstream end of the solid phase extraction column is in fluid communication with the sample inlet and the one or more reagent chambers, and further wherein the solid phase extraction column is oriented between the upper and lower disc plates such that a liquid passing therethrough travels in a direction perpendicular to the plane of the upper and lower disc plates; one or more serpentine microfluidic channels, wherein a downstream end of the solid phase extraction column is in fluid communication with the one or more serpentine channels; a waste chamber, wherein the waste chamber is disposed at a greater distance from the spinning axis of the rotatable disc than the sample inlet, and wherein the waste chamber is in fluid communication with the downstream end of a serpentine microfluidic channel; and a detection chamber, wherein the detection chamber is disposed at a greater distance from the spinning axis of the rotatable disc than the sample inlet, and wherein the detection chamber is in fluid communication with the downstream end of a serpentine microfluidic channel, the detection chamber containing a compound having the structure of Formula (II), or a salt thereof.

In particular embodiments, the compound of Formula (II) has the structure of Formula (I).

### BRIEF DESCRIPTION OF THE DRAWINGS

FIGS. 1a and 1b show the fluorescence response of caffeine orange (10 μM) with different concentration of caffeine in water under excitation of 530 nm. FIG. 1c shows the structure of Caffeine Orange. FIG. 1d shows pictures of caffeine orange (10 μM) aqueous solutions containing different concentrations of caffeine under the irradiation of a green laser beam, with the color of the laser beam transitioning from green at no to low concentration of caffeine to orange at higher concentrations of caffeine.

FIG. 2a demonstrates the selectivity of Caffeine Orange (10 μM) against different caffeine analogs (1 mM). FIG. 2b shows the fluorescence spectrum of Caffeine Orange (10 μM) incubated with different drink samples. FIG. 2c shows pictures of Caffeine Orange (10 μM) solutions containing eluents from different coffees (left: normal coffee; right: decaffeinated coffee) under irradiation of a green light laser pointer (532 nm). The caffeinated coffee exhibits an orange light under irradiation by a green laser pointer. The decaffeinated coffee exhibits a green light under irradiation by a green light laser pointer.

FIG. 3A is a scheme of the microfluidic disc for use in methods of automated solid phase extraction. Reference numeral 310 points to a sample chamber. Reference numeral 320 points to the inlet of the extraction chamber 330, which houses the solid phase extraction column. Reference numeral 340 points to the outlet of the extraction chamber, which leads to the serpentine channel 350. The serpentine channel terminates in a sample outlet or waste chamber 360. FIG. 3B shows a detailed scheme of a top-down view of the sample chamber 310, its inlet 320 into the top of the extraction chamber 330, and the outlet 340 from the bottom of the extraction chamber. In this scheme, fluid flows from “a” to “b”. FIG. 3C shows a side view of the extraction chamber. As fluid flows from “a” to “b”, it enters the top of the extraction chamber and flows down through the solid phase extraction column, which comprises supporting materials 370 as well as absorbent material 380.

FIG. 4 shows a scheme of the microfluidic disc for use in methods of automated solid phase extraction integrated with analyte detection. The disc contains a series of chambers connected by gated microfluidic channels, including a sample chamber 410, a pre-washing buffer chamber 420, a salt buffer chamber 430, a washing buffer chamber 440 and an elution buffer 450. Each of these chambers is connected by microfluidic channels to an extraction chamber 460, which houses the solid phase extraction column. The serpentine channel 470 leads from the extraction chamber to waste chamber 480, and an alternate serpentine channel leads to detection chamber 490.

FIG. 5 shows a scheme of a fluorescence detection module. Reference numeral 510 is a laser light source. 520 is a lens, 530 is a polarized filter that diffracts the laser light from 510, and 540 is a light detector.

FIG. 6A shows a photograph of a microfluidic disc for fully automated caffeine detection, and the enlarged area shows a detailed layout of the microfluidic disc. The number indicates the order of the valve operation and arrows indicate the flow of reagents. Valves 1, 3, 4, 6 and 7 are laser irradiated ferrowax microvalves that are usually closed and valves 2 and 5 are laser irradiated ferrowax microvalves that are usually open. FIG. 6B shows CCD images of the spinning disc at each reaction step. FIG. 6C shows the calibration curve obtained using the lab-on-a-disc and caffeine solution with known concentration. Each data point is an average of four samples tested with four different discs. FIG. 6D shows a comparison of caffeine concentration measured by fully automated lab-on-a-disc and syringe methods with real beverage samples: decaffeinated coffee (caffe vergnano 1882), Coca Cola, Red Bull energy drink, coffee (Angelinus Americano), and espresso (Nespresso Roma).

### DETAILED DESCRIPTION OF THE INVENTION

A description of example embodiments of the invention follows.

The present invention relates to compounds having the structure of Formula (I):

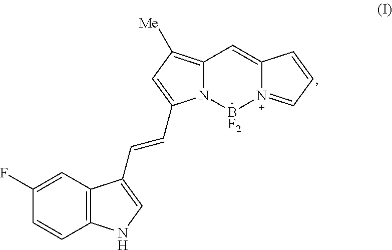

or salts thereof.

The compounds of the structure of Formula (I) and salts thereof are referred to herein as Caffeine Orange. Caffeine Orange, a new fluorescence sensor derived from the BODIPY scaffold, is highly selective against caffeine based upon the screening of around 100 structurally distinct analytes. The BODIPY scaffold shows outstanding photophysical properties, such as high extinction coefficient, high photostability and narrow emission bandwidth.⁴

Pharmaceutically acceptable salts of the compounds of the present invention are also included. For example, an acid salt of a compound of the present invention containing an amine or other basic group can be obtained by reacting the compound with a suitable organic or inorganic acid, resulting in pharmaceutically acceptable anionic salt forms. Examples of anionic salts include the acetate, benzenesulfonate; benzoate, bicarbonate, bitartrate, bromide, calcium edetate, camsylate, carbonate, chloride, citrate, dihydrochloride, edetate, edisylate, estolate, esylate, fumarate, glyceptate, gluconate, glutamate, glycollylarsanilate, hexylresorcinate, hydrobromide, hydrochloride, hydroxynaphthoate, iodide, isethionate, lactate, lactobionate, malate, maleate, mandelate, mesylate, methylsulfate, mucate, napsylate, nitrate, pamoate, pantothenate, phosphate/diphosphate, polygalacturonate, salicylate, stearate, subacetate, succinate, sulfate, tannate, tartrate, teoclate, tosylate, and triethiodide salts.

Salts of the compounds used in the kits, methods, and devices of the present invention containing a carboxylic acid or other acidic functional group can be prepared by reacting with a suitable base. Such a pharmaceutically acceptable salt may be made with a base which affords a pharmaceutically acceptable cation, which includes alkali metal salts (especially sodium and potassium), alkaline earth metal salts (especially calcium and magnesium), aluminum salts and ammonium salts, as well as salts made from physiologically acceptable organic bases such as trimethylamine, triethylamine, morpholine, pyridine, piperidine, picoline, dicyclohexylamine, N,N′-dibenzylethylenediamine, 2-hydroxyethylamine, bis-(2-hydroxyethyl)amine, tri-(2-hydroxyethyl)amine, procaine, dibenzylpiperidine, dehydroabietylamine, N,N′-bisdehydroabietylamine, glucamine, N-methylglucamine, collidine, quinine, quinoline, and basic amino acids such as lysine and arginine.

The synthesis of the compound of Formula (I) is described in Scheme 1, below, and in further detail in Example 1.

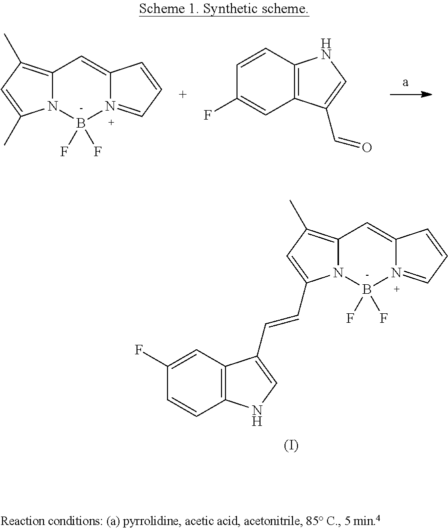

The invention further provides for kits for caffeine detection, comprising a compound of Formula (I) or a salt thereof, a reverse phase solid phase extraction column, and instructions indicating the use of the kit for the detection of caffeine.

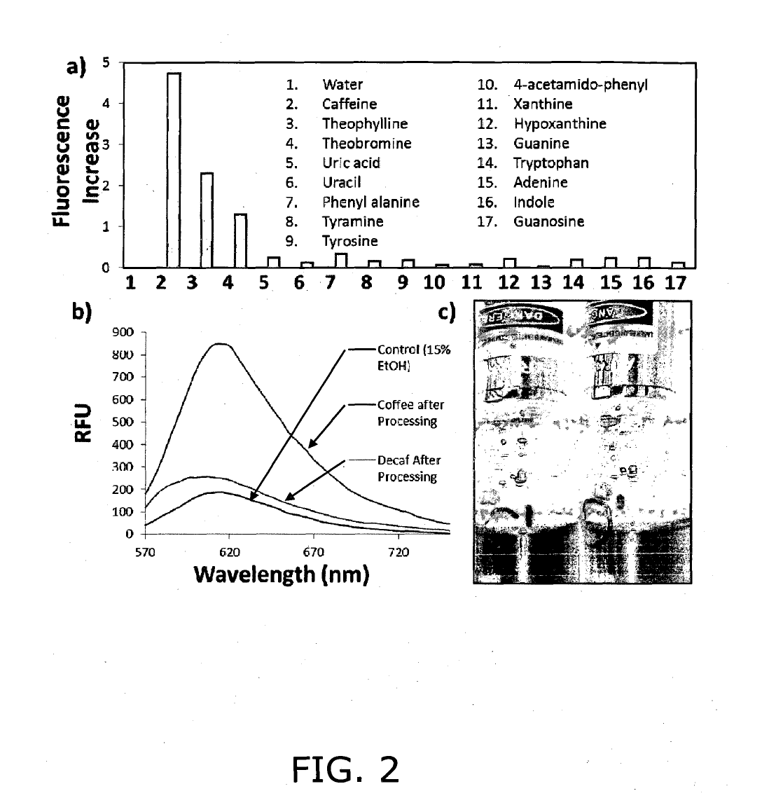

The kits described herein for the separation and detection of caffeine are portable. Through the usage of such reverse phase solid phase extraction materials, many of the interfering impurities are easily removed and caffeine can be efficiently concentrated for direct visualization. This visualization can be achieved by shining a laser pointer (532 nm, 5 mW) into the extracted coffee along with Caffeine Orange (FIGS. 2b and 2c, Example 2).

“Solid phase extraction”, or “SPE” is a separation process by which compounds that are dissolved or suspended in a liquid mixture are separated from other compounds in the mixture according to their chemical and/or physical properties. Typically, solid phase extraction utilizes a liquid mobile phase and a solid stationary phase. The solid stationary phase is alternately referred to herein as a “solid phase extraction column” or a “solid phase extraction cartridge”. If the compounds of interest in the liquid mixture are retained by the stationary phase, the stationary phase can be rinsed with an eluent to elute the compounds of interest. Solid phase extraction techniques are known to those of ordinary skill in the art. For example, Qu, J., Y. Qu, and R. M. Straubinger, Ultra-sensitive quantification of corticosteroids in plasma samples using selective solid-phase extraction and reversed-phase capillary high-performance liquid chromatography/tandem mass spectrometry. Anal Chem, 2007. 79(10): p. 3786-93; Batt, A. L., M. S. Kostich, and J. M. Lazorchak, Analysis of ecologically relevant pharmaceuticals in wastewater and surface water using selective solid-phase extraction and UPLC-MS/MS. Anal Chem, 2008. 80(13): p. 5021-30; and Chiuminatto, U., et al., Automated online solid phase extraction ultra high performance liquid chromatography method coupled with tandem mass spectrometry for determination of forty-two therapeutic drugs and drugs of abuse in human urine. Anal Chem, 2010. 82(13): p. 5636-45, the entire contents of which are incorporated herein by reference, use solid phase extraction to isolate analytes from biological samples.

Preferably, the solid phase extraction procedures used in the present invention are reverse phase solid phase extraction procedures, and the column for use in the methods and kits of the present invention is a reverse phase solid phase extraction column. “Reverse phase” as used herein, describes a solid stationary phase that is derivatized with hydrocarbon chains, such that compounds with mid- to low-polarity are retained on the solid phase extraction column, while compounds with higher polarity pass through the column. The compounds that are retained on the reverse phase solid phase extraction column may then be eluted by washing with an eluent of relatively low polarity. The reverse phase solid phase column materials utilize electrostatic, hydrophobic, and hydrophilic interactions to retain compounds of a certain polarity on the column, which allowing other compounds and solvents of another polarity to pass through the column without being retained. In certain embodiments, the kits and methods described herein utilize solid phase extraction columns that comprise reverse phase hydrocarbon-functionalized silanes, glass membranes, silica beads or polymer beads. Examples of materials that are used in reverse phase solid phase extraction columns include, but are not limited to silica based OROCHEM C2 SPE, OROCHEM C4 SPE, OROCHEM C8 SPE, OROCHEM C18 SPE, OROCHEM phenyl SPE, and OROCHEM cyclohexyl SPE (Orochem Technologies, Inc.). Preferably, the material is OROCHEM C4 SPE.

The kit further comprises instructions for use. The instructions for use can be in print format, for example as a brochure or illustrated pictorial guide, or alternately in digital format, for example on a USB drive or CD. The instructions for use contain a recitation of steps of the method that are further described in sections of the application, below, that pertain to methods of use of the compounds of Formula (I).

In certain embodiments, the kit further comprises a light source having a wavelength of about 532 nm. In further embodiments, the light source has a wavelength of about 495 nm to about 570 nm. In yet further embodiments, the light source has a wavelength of about 500 nm to about 560 nm, about 495 nm to about 550 nm, about 495 nm to about 540 nm, about 510 nm to about 560 nm, about 510 nm to about 550 nm, about 510 nm to about 540 nm, about 515 nm to about 550 nm, about 520 nm to about 540 nm, or about 528 nm to about 538 nm. Examples of light sources that may be included in kits of the invention include green laser light sources such as a green laser pointer.

All numeric values herein can be modified by the term “about”, whether or not explicitly indicated. The term “about” generally refers to a range of numbers that one of skill in the art would consider equivalent to the recited value (i.e., having the same function or result). In some versions the term “about” refers to ±10% of the stated value, ±8%, +7%, ±6%, ±5%, ±4%, or ±3% of the stated value. In other versions the term “about” refers to ±2% of the stated value. While compositions and methods are described in terms of “comprising” various components or steps (interpreted as meaning “including, but not limited to”), the compositions and methods can also “consist essentially of” or “consist of” the various components and steps, such terminology should be interpreted as defining essentially closed-member groups.

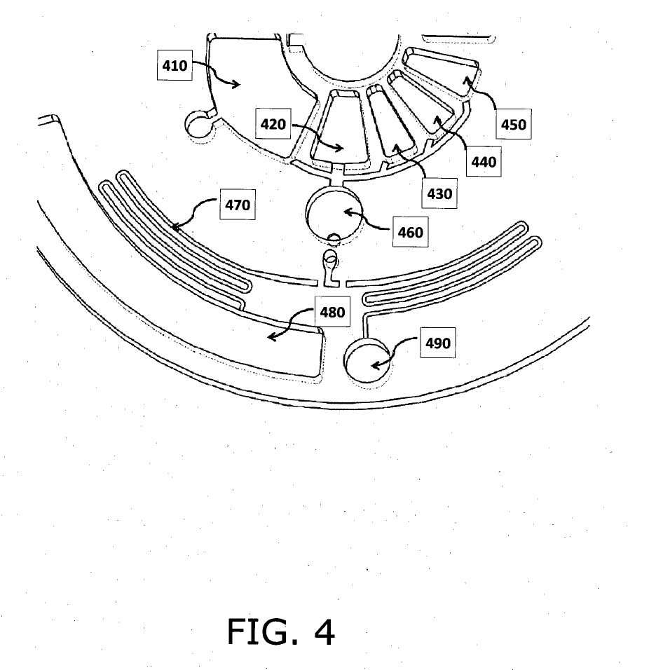

In further embodiments, the reverse phase solid phase extraction column of the kit is enclosed in a syringe. In yet further embodiments, the syringe is enclosed in a microfluidics device, is a described in detail below and in FIG. 4.

The invention described herein is based on the in vitro screening of a new fluorescence sensor derived from BODIPY scaffold, Caffeine Orange (Formula (I)), which is highly selective for the detection of caffeine.

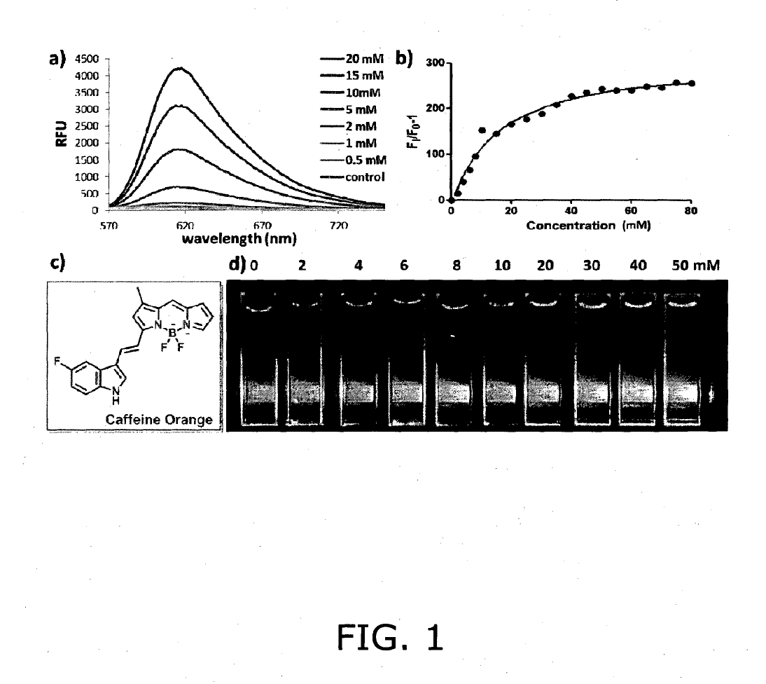

Caffeine Orange showed up to 66-fold fluorescence increase upon 20 mM of caffeine, with linear detection range of 0.05-100 mM of caffeine (FIGS. 1a and 1 b). To further elucidate the selectivity of Caffeine Orange, its response against 15 purine analogs was tested and it was proven to show better selectivity than most of the reported sensors (FIG. 2).

Previously reported caffeine sensors always encountered the problem of differentiating caffeine from theophylline and theobromine, two of its nearly identical analogs. In the case of Caffeine Orange, theobromine was successfully removed from the response list and theophylline showed less than half of caffeine response.

Accordingly, in another aspect, the present invention includes methods for the fluorescence-based selective detection of caffeine in a liquid medium. These methods comprise the steps of (a) loading a solid phase extraction column with a sample of a liquid medium thought to contain caffeine, such that caffeine, if present, is retained on the column and one or more impurities, if present, pass through the column; (b) contacting the solid phase extraction column loaded with the sample with one or more solutions sufficient to elute a solution thought to contain caffeine off of the column; (c) contacting the solution thought to contain caffeine of step (b) with a compound of Formula (I):

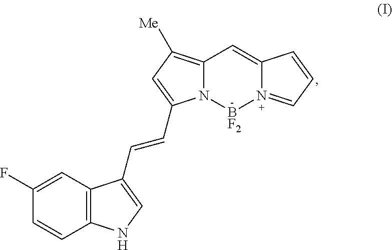

or a salt thereof;
to form an incubation media; (d) incubating the media of step (c) for a period of time sufficient to enable detection of caffeine by fluorescence if present in the solution;

and (e) detecting fluorescence in the incubated media, wherein a change in fluorescence signal as compared to a fluorescence signal of the compound of Formula (I) not in the presence of the solution thought to contain caffeine is indicative of the presence of caffeine in the liquid medium.

The step of loading an SPE column with a sample of a liquid medium, in this method or any other method of the invention disclosed herein, can occur through the use of a syringe, a pipet, an eye dropper, or any other liquid delivery device. Solid phase extraction columns for use with the methods of the invention have been described above.

A “liquid medium” as used herein, is a liquid that may include, but is not limited to, a food, a beverage, a medication, a cosmetic product, or a sample for laboratory analysis. The liquid medium may be a homogenous mixture such as a solution or a heterogeneous mixture or colloid. The SPE column separates impurities from caffeine by retaining caffeine, if present, on the SPE column while enabling impurities having a higher polarity than caffeine to elute through the column. In certain embodiments, such impurities comprise sugars, lipids, salts, proteins, tar, flavonoids, or other impurities that cannot be retained on the SPE column. In certain other embodiments, impurities are removed from caffeine because they cannot penetrate the SPE column.

After elution of the impurities, the SPE column in contacted with one or more solutions, resulting in an eluent thought to comprise caffeine. Solutions sufficient to elute caffeine off an SPE column include water, 5% ethanol in water, 10% ethanol in water, 15% ethanol in water, 20% ethanol in water, 25% ethanol in water and 30% ethanol in water. In preferred embodiments, the solution is about 15% ethanol in water.

The eluent, alternately referred to as the solution thought to contain caffeine, is contacted with a compound of Formula (I), or a salt thereof, and the mixture is subsequently incubated.

As used herein, “incubating” a sample means mixing a sample. Alternately, incubating means mixing and heating a sample. “Mixing” can comprise mixing by diffusion, or alternately by agitation of a sample. The conditions under which the mixture is incubated are sufficient to enable detection of caffeine by fluorescence, if caffeine is present in the mixture.

The incubated mixture is analyzed to detect fluorescence. Caffeine is determined to be present if a change in fluorescence signal is observed in the mixture, wherein the change is relative to a fluorescence signal of the compound of Formula (I) not in the presence of a solution of caffeine.

In some embodiments of the invention, “detecting fluorescence” means a quantitative analysis utilizing a fluorescence reader, fluorescence spectroscopy, fluorescence meter or another method that can quantify fluorescence. In alternate embodiments of the invention, “detecting fluorescence” means a qualitative visual analysis carried out by the human eye. In some embodiments of the invention, detecting fluorescence by visual analysis is carried out under visible light. In other embodiments of the invention, detecting fluorescence by visual analysis is carried out under certain wavelengths of light, e.g. about 365 nm (ultra-violet light), about 532 nm (green laser light). Fluorescence detection can be qualitative or quantitative.

As used herein, “spectroscopy” encompasses any method by which matter reacts with radiated energy. This includes, but is in no way limited to, microscopy, fluorescence microscopy, UV/Vis spectrometry, and flow cytometry.

A “change in fluorescence signal” as used herein, can be used to indicate a change in the fluorescence intensity of a sample after exposure to an analyte, as compared to a baseline exposure. For example, a fluorophore, such as a BODIPY-based fluorophore having the structure of Formula (I), exhibits a change in fluorescence intensity after exposure to an analyte such as caffeine. In some embodiments of the invention, the change in fluorescence intensity is an increase in fluorescence intensity. Alternately, a change in fluorescence can be a change in the wavelength of emitted light. For example, a change in wavelength may be observed as a change in the color of the fluorescence. A change in the color of the fluorescence can be a change in the color hue of the fluorescence (e.g. a green hue versus an orange hue), or can be a change in the tint or saturation of the fluorescence (e.g. a light pink versus a dark pink).

In certain embodiments, a change in the color of fluorescence is detectable under visible light, under a wavelength portion of the visible light spectrum, or under ultraviolet light.

In certain embodiments, under irradiation with a light source having a wavelength of about 532 nm, for example a green laser light pointer, an orange-colored fluorescence is indicative of the presence of caffeine in a solution.

In further embodiments, a change in fluorescence signal comprises a change in fluorescence intensity. In certain embodiments, a change in fluorescence intensity is an increase in fluorescence intensity.

In certain embodiments, the reverse phase solid phase extraction column that retains caffeine is enclosed in a syringe. In yet further embodiments, the syringe is enclosed in a microfluidics device, is a described in detail below and in FIG. 4.

The methods described herein are selective for the detection of caffeine over other possible analytes. The terms “selectivity” or “selective”, as used herein, refer to an analytical probe, for example a fluorescent dye, that produces a response for a target analyte that is distinguishable from responses of all other analytes. Selectivity can also refer to the analytical probe preferentially binding to a target analyte over all other analytes.

The terms “specificity” or “specific”, as used herein, refer to an analytical probe, for example a fluorescent dye, that produces a response for only one single analyte. Specificity can also refer to the analytical probe exclusively binding with a target analyte.

Another aspect of the invention relates to a fully integrated solid phase extraction technique on a microfluidic device with high efficiency and short operation time. The device described herein can handle real samples in a fully automated manner, and the methods utilizing such devices are advantageous for their high efficiency, low reagent consumption, few manual steps, high reproducibility, and short operation time.

As used herein, the term “microfluidic” refers to a device operating at or with or relating to volumes of fluids from 0.1 to 100 μL, preferably between 1 and 104. In some embodiments of the invention, a microfluidic device is a system flowing fluid in at least one solid phase extraction column, at least one channel, at least one chamber, at least one well and/or at least one port, each of which may be microfluidic.

In some embodiments of the invention, the microfluidics device further certain controls for its operation, such as actuating valves. Accordingly, in some embodiments, the microfluidics device further comprises microvalves that are actuated during operation of the device, for example by laser irradiation at a particular wavelength or by exposure to a heater, such as an infrared heater. The composition of the valve is inert to the solvents and the samples analyzed on the microfluidic device. In some aspects of the invention, the valve comprises ferrowax, a sol-gel composition, a hydrogel composition, a polymer film or an ice valve. Such microvalves function as gates between the channels of the microfluidic device and the chambers that hold, for example, a sample of liquid medium or an eluent used in washing the solid phase extraction column. Actuation of the microvalves in an intended sequence enables isolating of a analyte solution from the sample of liquid medium. In certain embodiments, actuation of the microvalves in an intended sequence enables isolation of a solution comprising caffeine. The microfluidics devices for use in the invention, in use, spin on an axis in a manner analogous to a centrifuge.

As used herein, “fluid” refers to both a gas or a liquid.

In one aspect, the invention is a centrifugal microfluidic device, comprising an upper disc plate, a lower disc plate, a sample inlet, one or more reagent chambers, an extraction chamber comprising a solid phase extraction column, one or more serpentine microfluidic channels, a waste chamber and a detection chamber. In certain embodiments, this microfluidic device is alternately referred to as a centrifugal microfluidic disc.

The one or more reagent chambers each independently contain a reagent liquid. The upstream end of the solid phase extraction column is in fluid communication with the sample inlet and the one or more reagent chambers. The downstream end of the solid phase extraction column is in fluid communication with the one or more serpentine channels.

The solid phase extraction column is oriented between the upper and lower disc plates such that a liquid passing through the column travels in a direction perpendicular to the plane of the upper and lower disc plates. In certain embodiments, the solid phase extraction column is a reverse phase solid phase extraction column. Example embodiments of solid phase extraction column materials are described above.

The waste chamber is disposed at a greater distance from the spinning axis of the rotatable disc than the sample inlet. As the microfluidic device spins on its spinning axis, a liquid sample introduced into the device at the sample inlet moves radially outward, for example through microfluidic channels, toward the waste chamber. The waste chamber is in fluid communication with the downstream end of a serpentine microfluidic channel.

The detection chamber is disposed at a greater distance from the spinning axis of the rotatable disc than the sample inlet. As the microfluidic device spins on its spinning axis, a liquid sample introduced into the device at the sample inlet moves radially outward, for example through microfluidic channels, toward the detection chamber. The detection chamber is in fluid communication with the downstream end of a serpentine microfluidic channel. The detection chamber contains a compound having the structure of Formula (II):

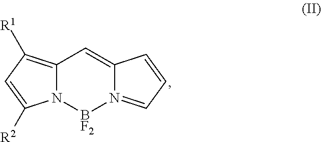

or salts thereof;

wherein R¹ is C₁-C₁₂ alkyl; and

R² is C₁-C₆ alkyl or C₂-C₆ alkenyl, optionally substituted with C₆-C₁₄ aryl or C₃-C₁₃ heteroaryl.

Example BODIPY-based compounds of Formula (II) that may be used as fluorophores in methods of the present invention may be found in Lee, J. S. et al. “Synthesis of a bodipy library and its application to the development of live cell glucagon imaging probe” J. Am. Chem. Soc. 2009, 131, 10077. In preferred embodiments of the invention, the fluorophore is a compound having the structure of Formula (I):

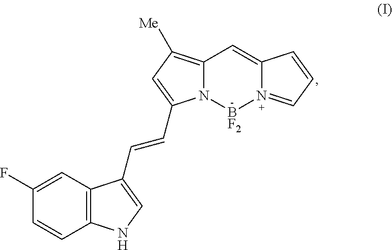

or a salt thereof.

In certain embodiments of the invention, microfluidics device comprises a detection chamber storing a solution comprising a compound of Formula (II) and a microvalve that opens to enable mixing of the analyte solution and the solution comprising a compound of Formula (II). An example embodiment is depicted in FIG. 4. A microvalve is alternately referred to herein as a valving unit.

In another aspect, the invention relates to methods for the fluorescence-based selective detection of an analyte in a liquid medium on a microfluidic disc utilizing a fluorophore of Formula (II):

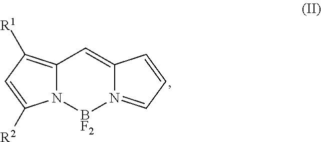

or salts thereof;

wherein R¹ is C₁-C₁₂ alkyl; and

R² is C₁-C₆ alkyl or C₂-C₆ alkenyl, optionally substituted with C₆-C₁₄ aryl or C₃-C₁₃ heteroaryl.

The method comprises providing a rotatable microfluidic disc as described above and loading a liquid medium thought to contain the analyte into the sample inlet of the microfluidic disc.

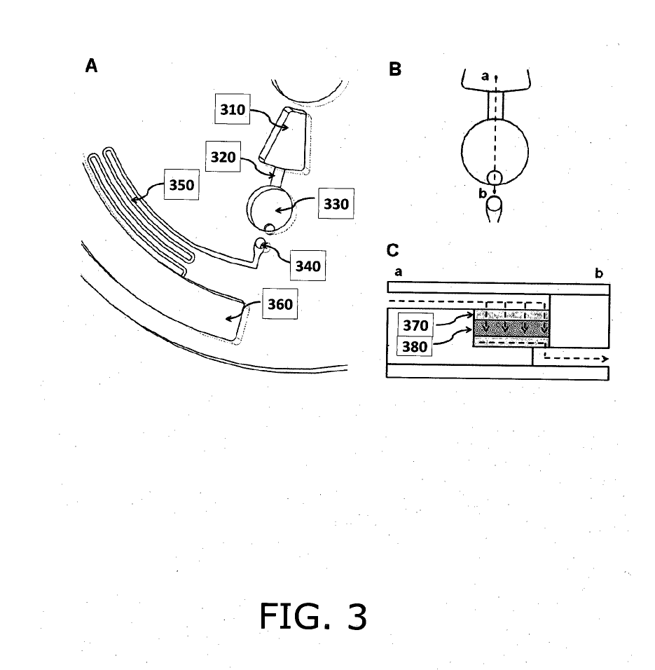

The methods further comprise rotating the disc such that centrifugal force causes the liquid medium to travel from the sample inlet through the solid phase extraction column into the sample outlet, such that the analyte, if present in the sample, is retained on the SPE column while any one or more impurities pass through the column and into the waste chamber. Liquid flow through the SPE column occurs in a direction perpendicular to the direction of radial force. The direction of liquid flow through the SPE is also described as being perpendicular to the plane of the upper and lower disc plates. This is depicted in FIG. 3C.

The methods further comprise contacting the solid phase extraction column with one or more reagent liquids from one or more reagent chamber. In certain embodiments, the one or more reagent liquids elute further impurities off of the column. In other embodiments, one or more reagent liquids are sufficient to elute a solution thought to contain the analyte off of the column. In further embodiments, the one or more reagent chambers each contain a reagent liquid, each independently selected from a pre-washing buffer, a salt buffer, a washing buffer, an elution buffer, a blocking buffer or a detection solution. Example reagent liquids sufficient to elute an analyte off an SPE column include water, 5% ethanol in water, 10% ethanol in water, 15% ethanol in water, 20% ethanol in water, 25% ethanol in water and 30% ethanol in water. In preferred embodiments, the reagent liquid is about 15% ethanol in water.

The methods further comprise contacting the solution thought to contain the analyte with the fluorophore of Formula (II) in the detection chamber of the microfluidics device to form an incubation media, then incubating the media for a period of time sufficient to enable detection of the analyte by fluorescence, if the analyte is present in the solution.

The methods further comprise detecting fluorescence in the incubated media, wherein a change in fluorescence signal as compared to a fluorescence signal of the fluorophore of Formula (II) not in the presence of the solution thought to contain the analyte is indicative of the presence of the analyte in the liquid medium.

In further embodiments, a change in fluorescence signal is a change in the color of fluorescence, a change in fluorescence intensity, or a combination thereof.

In further embodiments, the method further comprises controlling the flow resistance by directing liquid flow through the serpentine channel, thereby altering the elution time of the caffeine into the sample outlet.

In certain embodiments of the invention, the microfluidics device further comprises microvalves that are actuated during operation of the device. Accordingly, in certain embodiments, an actuation of at least one valving unit manipulates a flow or flow path of the liquid medium, a flow or a flow path of the reagent liquid, or a combination thereof. In certain embodiments, the flow of the reagent liquid is manipulated by an actuation of at least one valving unit.

Microvalve compositions and actuation of microvalves are detailed above. In certain embodiments, the valving unit comprises a phase transition valve that is actuated by laser irradiation or heat. In further embodiments, the phase transition valve comprises ferrowax, hydrogel, sol-gel, ice or a polymer film.

In particular embodiments, the analyte is caffeine. In yet more particular embodiments, the fluorophore of Formula (II) is the compound of Formula (I), Caffeine Orange. In certain embodiments, caffeine is determined to be present in a liquid medium when an orange colored fluorescence is observed under irradiation with a light source having a wavelength of about 532 nm and when a fluorophore of Formula (I) is utilized.

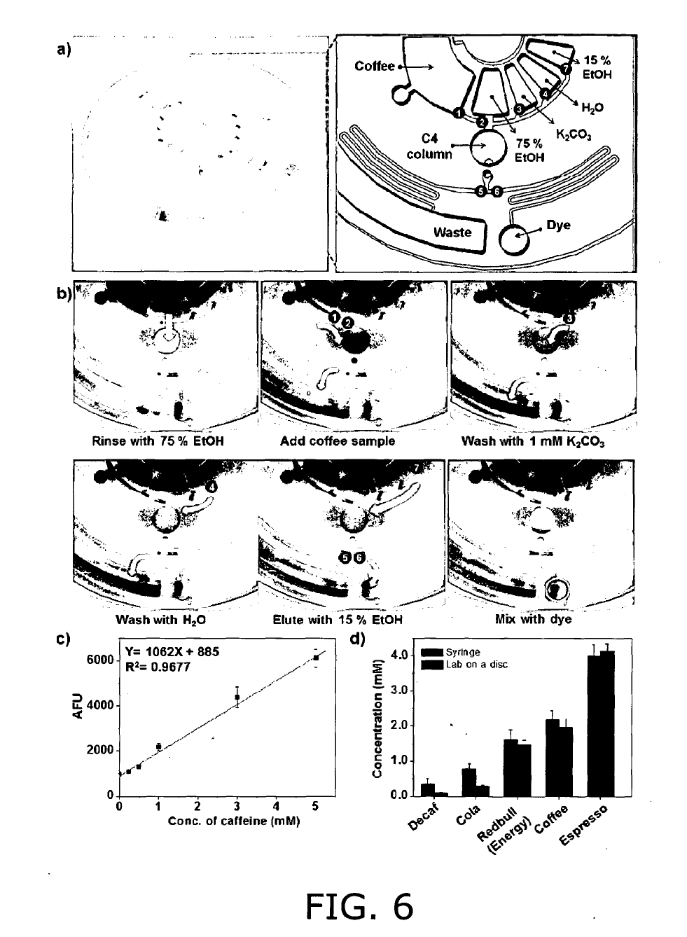

Described herein are methods for the selective fluorescence-based detection of caffeine automated in a microfluidic device system. Such a system is advantageous in providing ease of operation and consistency in experimental set up. Microfluidic techniques, previously applied to separate blood and DNA and materials containing complicated matrices, are used herein in a novel application of separating caffeine from beverages or other consumer products.⁶ This process is depicted in FIG. 6.

In a preferred embodiment, the fully integrated solid phase extraction and caffeine detection module is illustrated in FIG. 4. All fluidic flow is propelled by centrifugal force induced by rotation of body and is controlled by actuating valves. Also, in order to provide enough retention time of each solution in packed sorbent, the outlets, specifically the waste chamber and the detection chamber, are paired with a serpentine channel 470. In use, the following operations are performed on the microfluidics disc:

1. The sorbent is washed by pre-washing buffer from chamber 420.
2. Sample solution from chamber 410 is moved to extraction chamber 460 and flowed through the packed sorbent.
3. The sorbent absorbing the target analytes is washed to remove the residue with salt buffer from 430 and washing buffer from 440.
4. The fluidic path is changed from waste chamber 480 to detection chamber 490 containing detection dye.
5. Elution buffer from 450 desorbs analytes from the solid surface transferring to detection chamber.
6. Fluorescence signal is measured.

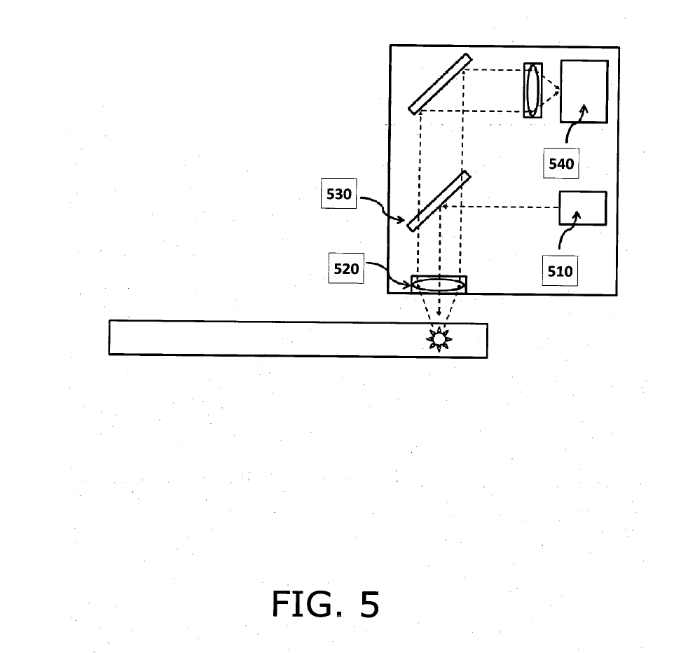

In a preferred embodiment of the invention, fluorescence is measured with a detection module as depicted in FIG. 5. A laser light source 510 is irradiated and diffracted by polarized filter 530 to apply the light on a fluorophore in microfluidic device. Then, emitted lights from the sample are collimated by the lens 520 and diffracted again toward light detector 540. The light detector converts collected light to electrical signals.

In another aspect, the invention relates to methods for solid phase extraction of an analyte from a liquid medium on a microfluidic disc. In the present invention, a sample of a liquid medium passes through a solid phase extraction column under centrifugal force such that the analyte is maintained on the column. One or more solutions are then utilized to remove the analyte from the column, enabling collection of the analyte at a sample outlet on the microfluidic disc. In some embodiments of the invention, the one or more solutions are stored in chambers on the microfluidic disc. The disc optionally comprises a serpentine channel downstream from the solid phase extraction column, which can be used to resist liquid flow on the disc, thereby enabling control over the elution time of the analyte.

The methods for solid phase extraction of an analyte comprise providing a rotatable microfluidic disc, the disc comprising a sample inlet, an extraction chamber comprising a solid phase extraction column and a sample outlet; loading a liquid medium thought to contain an analyte into the sample inlet; and rotating the disc such that centrifugal force causes the liquid medium to travel from the sample inlet through the solid phase extraction column into the sample outlet, such that the analyte, if present, is retained on the column.

Furthermore, in the rotatable microfluidic disc, an upstream end of the solid phase extraction column is in fluid communication with the sample inlet, and a downstream end of the solid phase extraction column is in fluid communication with the sample outlet. The sample outlet is disposed at a greater distance from the spinning axis of the rotatable disc than the sample inlet.

In certain embodiments, the microfluidic disc further comprises an upper disc plate, a lower disc plate, optionally a serpentine microfluidic channel, wherein a downstream end of the solid phase extraction column is in fluid communication with the serpentine channel and further wherein a downstream end of the optional serpentine microfluidic channel is in fluid communication with the sample outlet.

Liquid flow through the SPE column occurs in a direction perpendicular to the direction of radial force. The direction of liquid flow through the SPE is also described as being perpendicular to the plane of the upper and lower disc plates. This is depicted in FIG. 3C. Example embodiments of SPE columns are described above.

In certain embodiments, the microfluidic disc further comprises one or more reagent chambers containing a reagent liquid, each independently selected from a pre-washing buffer, a salt buffer, a washing buffer, an elution buffer, a blocking buffer or a detection solution. Example reagent liquids sufficient to elute an analyte off an SPE column include water, 5% ethanol in water, 10% ethanol in water, 15% ethanol in water, 20% ethanol in water, 25% ethanol in water and 30% ethanol in water. In preferred embodiments, the reagent liquid is about 15% ethanol in water.

In further embodiments, the methods further comprise the step of eluting the analyte from the solid phase extraction column by contacting the column with an elution buffer, wherein the step of eluting is performed after retention of the analyte on the SPE column.

In yet further embodiments, the methods further comprise controlling flow resistance by directing liquid flow through the serpentine channel, thereby altering the elution time of the analyte into the sample outlet.

An example embodiment of a microfluidics device used in methods for solid phase extraction of an analyte from a liquid medium is depicted in FIG. 3. A sample solution containing at least one kind of target is introduced to sample chamber 310. Then, solution is transferred through inlet channel 320 to extraction chamber 330 incorporating the extraction column comprising the absorbent 380 and supporting materials 370 to pack the absorbent. Fluid can be transferred in the radial direction by applying the centrifugal force based-pressure induced by rotation of the disc. Then, solution is moved to waste chamber 360 through the outlet 340 of the extraction chamber. To control the retention time of sample solution in extraction chamber, serpentine channel 350 is employed to control the flow resistance of channel. In the extraction chamber, packing materials for solid phase extraction are packed between supporting fits which are located between top and bottom discs. Therefore, flow velocity is smaller than that of the conventional packed beads in microchannels because the fluid transfer is made in flow-through mode. (FIGS. 3B-3C).

### EXAMPLES

### Materials and Methods

All reactions were performed in oven-dried glassware under a positive pressure of nitrogen. Unless otherwise noted, starting materials and solvents were purchased from Aldrich and Acros Organics and used without further purification. Analytical TLC was carried out on Merck 60 F254 silica gel plate (0.25 mm layer thickness) and visualization was done with UV light. Column chromatography was performed on Merck 60 silica gel (230-400 mesh). NMR spectra were recorded on a Bruker Avance 300 NMR spectrometer. Chemical shifts are reported as δ in units of parts per million (ppm) and coupling constants are reported as a J value in Hertz (Hz). Mass of all the compounds was determined by LC-MS of Agilent Technologies with an electrospray ionization source. All fluorescence assays were performed with a Gemini XS fluorescence plate reader.

### Example 1

### Chemical Synthesis of Caffeine Orange

Synthesis of Caffeine Orange (C₂₀H₁₅BF₃N₃; m/z 365.13): 2,4-dimethyl pyrrole⁴ (15 mg, 68 μmol) and aldehyde (136 μmol, 2 equiv) were dissolved in acetonitrile, with 6 equiv of pyrrolidine (48 μL) and 6 equiv of acetic acid (32 μL). The mixture was reacted at 85° C. for 5 min. The reaction mixture was then cooled down to rt, and then monitored by TLC. The resulting crude mixtures were concentrated under vacuum and purified by column to get 10 mg solid (yield: 40%).

### Example 2

### Caffeine Separation and Visualization Using Reverse Phase SPE

The SPE syringe was prepared by inserting reverse phase gel material (OROCHEM 3 mL C4 SPE cartridge, 200 mg material) into a BRAUN Injekt® 5 mL/Luer Solo syringe. The syringe was first blocked with one frit (Catalog: 211408) and after inputting the gel material, another frit was inserted to cover the top. The whole syringe was packed tight.

The reverse phase SPE was rinsed with 75% EtOH in H₂O (2 mL) and then 5 mL coffee was pushed through the SPE cartridge to collect caffeine on the SPE. The SPE column was washed sequentially with 1 mM K₂CO₃ (1 mL) and H₂O (1 mL), then was eluted with 15% EtOH in H₂O (1 mL). The eluent was collected into a glass tube containing 15 uL 1 mM dye solution. The solution was mixed and visualized with a green laser pointer (532 nm, 5 mW, Aurora).

### Example 3

### Caffeine Separation and Visualization Using Reverse Phase Microfluidics Device

The disc (dia.=12 cm) was equipped with chambers for coffee sample (1.2 mL), 75% EtOH (400 μL), K₂CO₃ (200 μL), DI water (200 μL), 15% EtOH (200 μL), and caffeine orange (0.1 mM, 22 μL). The microfluidic channels and chambers were fabricated by CNC-micromachining and the device was composed of three pieces of polycarbonate disc. The 5 mm thick middle disc had a through-hole for a C4 column, which was prepared by packing the C4 particles between the fits. The top disc had sample injection holes and the ferrowax microvalves were actuated on demand by laser irradiation. As the disc spun (3000 rpm, 1 min), 75% EtOH solution was transferred to the C4 column while big particles in the coffee sample sedimented in the sample chamber. After opening valve #1 by laser irradiation, 1 mL of supernatant particle-free coffee sample was transferred into C4 column chamber and the input channel was blocked by closing the valve #2. Then, the C4 column was washed by K₂CO₃ and DI water by actuation of the valves #3 and #4, respectively. Then, the channel to the waste chamber was closed by the laser irradiation on the valve #5 and the caffeine was eluted and transferred to the detection chamber by the actuation of the vales #5 and #6. The eluted caffeine was mixed with pre-stored Caffeine Orange and the final concentration was measured under excitation at 532 nm with an optical fiber-coupled spectrophotometer.

### REFERENCES

1. Hurst, W. J.; Martin, Jr., R. A.; Tarka, Jr., S. M. In Caffeine; Spiller, G. A., Ed.; CRC Press: Boca Raton, 1998; pp 13-33.
2. (a) Waldvogel, S. R.; Froehlich, R.; Schalley, C. A. Angew. Chem. Int. Ed. 2000, 39, 2472-2475. (b) Fiammengo, R; Crego-Calama, M.; Timmerman, P.; Reinhoudt, D. N. Chem. Eur. J. 2003, 9, 784-792. (c) Goswami, S.; Mahapatra, A. K.; Mukherjee, R. J. Chem. Soc. Perkin. Trans. 1, 2001, 2717-2726. (d) Rochat S., Steinmann S. N., Corminboeuf C. and Severin K., Chem. Commun., 2011, 47, 10584-10586
3. (a) Valeur, B.; Leray, I. Coord. Chem. Rev. 2000, 205, 3-40 (b) deSilva, A. P.; Gunaratne, H. Q. N.; Gunnlaugsson, T.; Huxley, A. J. M.; McCoy, C. P.; Rademacher, J. T.; Rice, R. E. Chem. Rev. 1997, 97, 1515-1566 (c) Czarnik, A. W. Top. Curr. Chem. 1994, 561, 314-323 (d) Ait-Haddou, H.; Wiskur, S. L.; Lynch, V. M.; Anslyn, E. V. J. Am. Chem. Soc. 2001, 123, 11296-11297 (e) Zhao, J. Z; James, T. D. Chem. Comm. 2005, 1889-1891 (f) Kubo, Y.; Kobayashi, A.; Ishida, T.; Misawa, Y.; James. T. D. Chem. Comm. 2005, 2846-2848 (g) Maue, M.; Schrader, T. Angew. Chem. Int. Ed. 2005, 44, 2265-2270
4. Lee, J. S.; Kang, N. Y.; Kim, Y. K.; Samanta, A.; Feng, S.; Kim, H. K.; Vendrell, M.; Park, J. H.; Chang, Y. T. J. Am. Chem. Soc. 2009, 131, 10077-10082
6. J. Park, V. Sunkara, T. H. Kim, H. Hwang, Y. K. Cho, Anal Chem 2012, 84, 4634-4634

The teachings of all patents, published applications and references cited herein are incorporated by reference in their entirety.

While this invention has been particularly shown and described with references to example embodiments thereof, it will be understood by those skilled in the art that various changes in form- and details may be made therein without departing from the scope of the invention encompassed by the appended claims.

1. A kit for the detection of caffeine in a sample, comprising:
a reverse phase solid phase extraction column;
a compound having the structure of Formula (I): 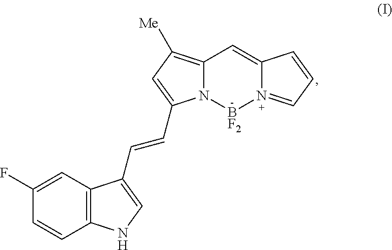
or a salt thereof; and
instructions indicating the use of the kit for the detection of caffeine in a sample.

2. The kit of claim 1, further comprising a light source having a wavelength of about 532 nm.

3. The kit of claim 1, wherein the reverse phase solid phase extraction column is enclosed in a syringe.

4. A compound having the structure of Formula (I): 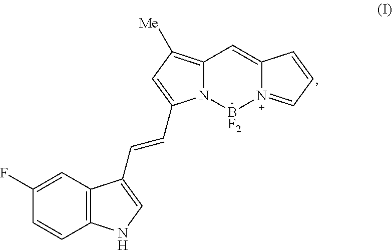
or a salt thereof.

5. A method for the fluorescence-based selective detection of caffeine in a liquid medium, comprising:
(a) loading a solid phase extraction column with a sample of a liquid medium thought to contain caffeine, such that caffeine, if present, is retained on the column and one or more impurities, if present, pass through the column;
(b) contacting the solid phase extraction column loaded with the sample with one or more solutions sufficient to elute a solution thought to contain caffeine off of the column;
(c) contacting the solution thought to contain caffeine of step (b) with a compound of Formula (I) of claim 4: 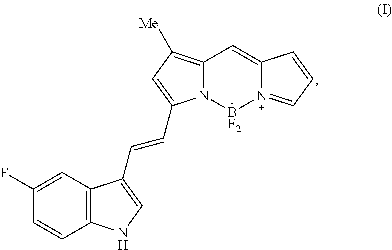
or a salt thereof;
to form an incubation media;
(d) incubating the media of step (c) for a period of time sufficient to enable detection of caffeine by fluorescence if present in the solution; and
(e) detecting fluorescence in the incubated media, wherein a change in fluorescence signal as compared to a fluorescence signal of the compound of Formula (I) not in the presence of the solution thought to contain caffeine is indicative of the presence of caffeine in the liquid medium.

6. The method of claim 5, wherein detecting fluorescence in the incubated media comprises qualitative visual analysis or analysis by fluorescence reader, fluorescence meter or fluorescence spectroscopy.

7. The method of claim 5, wherein the change in fluorescence comprises a change in the color of the fluorescence.

8. The method of claim 7, wherein the change in the color of the fluorescence is detectable under visible light or a wavelength portion thereof or ultraviolet light.

9. The method of claim 8, wherein under irradiation with a light source having a wavelength of about 532 nm, an orange-colored fluorescence is indicative of the presence of caffeine in the liquid medium.

10. The method of claim 5, wherein the change in fluorescence comprises a change in fluorescence intensity.

11. (canceled)

12. The method of claim 5, wherein the solid phase extraction column is enclosed in a syringe.

13. The method of claim 5, wherein the solid phase extraction column is a component of a microfluidics device.

14. A method for solid phase extraction of an analyte from a liquid medium on a microfluidic disc, the method comprising:
(a) providing a rotatable microfluidic disc, the disc comprising:
a sample inlet;
an extraction chamber comprising a solid phase extraction column, wherein an upstream end of the solid phase extraction column is in fluid communication with the sample inlet, and
a sample outlet, wherein a downstream end of the solid phase extraction column is in fluid communication with the sample outlet, and further wherein the sample outlet is disposed at a greater distance from the spinning axis of the rotatable disc than the sample inlet;
(b) loading a liquid medium thought to contain an analyte into the sample inlet; and
(c) rotating the disc such that centrifugal force causes the liquid medium to travel from the sample inlet through the solid phase extraction column into the sample outlet, such that the analyte, if present, is retained on the column, wherein liquid flow through the solid phase extraction column occurs in a direction perpendicular to the direction of radial force.

15. The method of claim 14, wherein the disc further comprises:
an upper disc plate;
a lower disc plate;
wherein the solid phase extraction column is oriented between the upper and lower disc plates such that a liquid passing therethrough travels in a direction perpendicular to the plane of the upper and lower disc plates;
optionally a serpentine microfluidic channel, wherein a downstream end of the solid phase extraction column is in fluid communication with the serpentine channel; and
further wherein a downstream end of the optional serpentine microfluidic channel is in fluid communication with the sample outlet.

16. The method of claim 15, wherein the disc further comprises one or more reagent chambers containing a reagent liquid, each independently selected from a pre-washing buffer, a salt buffer, a washing buffer, an elution buffer, a blocking buffer or a detection solution.

17. The method of claim 16, further comprising the step of eluting the analyte from the solid phase extraction column by contacting the column with an elution buffer, wherein the step of eluting is performed after step (c).

18. The method of claim 17, further comprising controlling flow resistance by directing liquid flow through the serpentine channel, thereby altering the elution time of the analyte into the sample outlet.

19.-20. (canceled)

21. A method for fluorescence-based selective detection of an analyte in a liquid medium on a microfluidic disc, the method comprising:
(a) providing a rotatable microfluidic disc, the disc comprising:
an upper disc plate;
a lower disc plate;
a sample inlet;
one or more reagent chambers, each independently containing a reagent liquid;
an extraction chamber comprising a solid phase extraction column, wherein an upstream end of the solid phase extraction column is in fluid communication with the sample inlet and the one or more reagent chambers, and further wherein the solid phase extraction column is oriented between the upper and lower disc plates such that a liquid passing therethrough travels in a direction perpendicular to the plane of the upper and lower disc plates;
one or more serpentine microfluidic channels, wherein a downstream end of the solid phase extraction column is in fluid communication with the one or more serpentine channels;
a waste chamber, wherein the waste chamber is disposed at a greater distance from the spinning axis of the rotatable disc than the sample inlet, and wherein the waste chamber is in fluid communication with the downstream end of a serpentine microfluidic channel; and
a detection chamber, wherein the detection chamber is disposed at a greater distance from the spinning axis of the rotatable disc than the sample inlet, and wherein the detection chamber is in fluid communication with the downstream end of a serpentine microfluidic channel, the detection chamber containing a fluorophore of the structure of Formula (II); 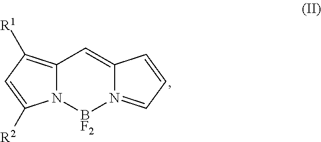
or a salt thereof;
wherein R¹ is C₁-C₁₂ alkyl; and
R² is C₁-C₆ alkyl or C₂-C₆ alkenyl, optionally substituted with C₆-C₁₄ aryl or C₃-C₁₃ heteroaryl;
(b) loading a liquid medium thought to contain the analyte into the sample inlet;
(c) rotating the disc such that centrifugal force causes the liquid medium to travel from the sample inlet through the solid phase extraction column into the sample outlet, such that the analyte, if present, is retained on the column, and one or more impurities, if present, pass through the column and into the waste chamber, wherein liquid flow through the solid phase extraction column occurs in a direction perpendicular to the direction of radial force;
(d) contacting the solid phase extraction column with one or more reagent liquids from one or more reagent chambers, wherein at least one of the one or more reagent liquids is sufficient to elute a solution thought to contain the analyte off of the column;
(e) contacting the solution thought to contain the analyte of step (d) with the fluorophore of Formula (II) in the detection chamber to form an incubation media;
(f) incubating the media of step (e) for a period of time sufficient to enable detection of the analyte by fluorescence if present in the solution; and
(g) detecting fluorescence in the incubated media, wherein a change in fluorescence signal as compared to fluorescence of the fluorophore of Formula (II) not in the presence of the solution thought to contain the analyte is indicative of the presence of the analyte in the liquid medium.

22.-34. (canceled)

35. The method of claim 21, wherein the fluorophore is a compound having the structure of Formula (I): 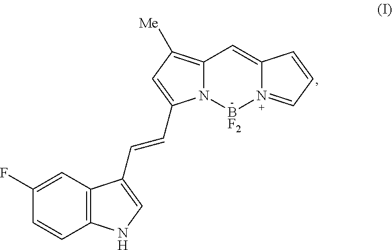
or a salt thereof.

36. (canceled)

37. A centrifugal microfluidic device, comprising:
an upper disc plate;
a lower disc plate;
a sample inlet;
one or more reagent chambers, each independently containing a reagent liquid;
an extraction chamber comprising a solid phase extraction column, wherein an upstream end of the solid phase extraction column is in fluid communication with the sample inlet and the one or more reagent chambers, and further wherein the solid phase extraction column is oriented between the upper and lower disc plates such that a liquid passing therethrough travels in a direction perpendicular to the plane of the upper and lower disc plates;
one or more serpentine microfluidic channels, wherein a downstream end of the solid phase extraction column is in fluid communication with the one or more serpentine channels;
a waste chamber, wherein the waste chamber is disposed at a greater distance from the spinning axis of the rotatable disc than the sample inlet, and wherein the waste chamber is in fluid communication with the downstream end of a serpentine microfluidic channel; and
a detection chamber, wherein the detection chamber is disposed at a greater distance from the spinning axis of the rotatable disc than the sample inlet, and wherein the detection chamber is in fluid communication with the downstream end of a serpentine microfluidic channel, the detection chamber containing a compound having the structure of Formula (II): 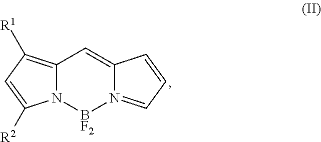
or a salt thereof;
wherein R¹ is C₁-C₁₂ alkyl; and
R² is C₁-C₆ alkyl or C₂-C₆ alkenyl, optionally substituted with C₆-C₁₄ aryl or C₃-C₁₃ heteroaryl.

38. The device of claim 37, wherein the compound has the structure of Formula (I): 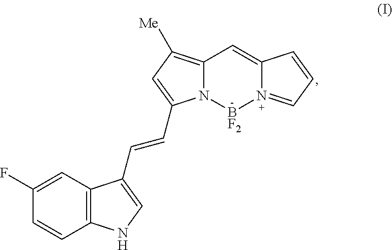
or a salt thereof.
